(19) United States
(12) Patent Application Publication
(10) Pub. No.: US 2001/0051783 A1
(43) Pub. Date: Dec. 13, 2001

(54) METHOD AND APPARATUS FOR TREATMENT OF AIR WAY OBSTRUCTIONS

(76) Inventors: STUART D. EDWARDS, PORTOLA VALLEY, CA (US); RONALD G. LAX, PALM CITY, FL (US)

Correspondence Address: WILSON SONSINI GOODRICH & ROSATI, 650 PAGE MILL ROAD, PALO ALTO, CA, 943041050

(21) Appl. No.: 08/895,783

(22) Filed: Jul. 17, 1997

Related U.S. Application Data
(63) Continuation-in-part of application No. 08/642,327, filed on May 3, 1996, now Pat. No. 5,807,308.
Application No. 08/642,327 is a continuation-in-part of application No. 08/606,195, filed on Feb. 23, 1996, now Pat. No. 5,707,349.

(51) Int. Cl. A61B 17/20; A61J 7/00

(52) U.S. Cl. 604/22; 604/77

(57) ABSTRACT

An apparatus reduces the volume of selected sections of a tongue. The apparatus includes an introducer means. An energy delivery device means is at least partially positioned in an interior of the introducer means. The energy delivery device means is configured to deliver sufficient energy to ablate an interior of the tongue without damaging a main branch of the hypoglossal nerve of the tongue. An energy delivery device advancement and retraction means is coupled to the energy delivery device means to advance and retract at least a portion of the energy delivery device means in and out of a selected tongue surface. A cabling means is coupled to the energy delivery device means.

### CROSS-REFERENCE TO RELATED APPLICATIONS

This application is a continuation-in-part application of U.S. patent application Ser. No. 08/642,327, filed May 3, 1996, which is a continuation-in-part of U.S. patent appplication Ser. No. 08/606,195, filed Feb. 23, 1996, which cross-references U.S. patent application Ser. No. 08/516,781 filed Aug. 18, 1995, having named inventors Stuart D. Edwards, Edward J. Gough and David L. Douglass, which is a continuation-in-part of U.S. application Ser. No. 08/239,658, filed May 9, 1994. This application is also related to U.S. patent application Ser. No. 08/642,053, filed May 3, 1996, all applications of which are incorporated by reference herein.

### BACKGROUND OF THE INVENTION

1. Field of the Invention

This invention relates to a method and apparatus for improving upper airway patency in human patients, and more particularly to a method and apparatus which utilizes an energy delivery device energy to create cell necrosis in selected sections of the tongue and/or lingual tonsil without damaging the main branches of the hypoglossal nerve.

2. Description of Related Art

Sleep-apnea syndrome is a medical condition characterized by daytime hypersomnomulence, morning arm aches, intellectual deterioration, cardiac arrhythmias, snoring and thrashing during sleep. It is caused by frequent episodes of apnea during the patient's sleep. The syndrome is classically subdivided into two types. One type, termed “central sleep apnea syndrome”, is characterized by repeated loss of respiratory effort. The second type, termed obstructive sleep apnea syndrome, is characterized by repeated apneic episodes during sleep resulting from obstruction of the patient's upper airway or that portion of the patient's respiratory tract which is cephalad to, and does not include, the larynx.

Treatment thus far includes various medical, surgical and physical measures. Medical measures include the use of medications such as protriptyline, medroxyprogesterone, acetazolamide, theophylline, nicotine and other medications in addition to avoidance of central nervous system depressants such as sedatives or alcohol. The medical measures above are sometimes helpful but are rarely completely effective. Further, the medications frequently have undesirable side effects.

Physical measures have included weight loss to open the airway and use of nasal CPAP and various tongue retaining devices. These devices may be partially effective but are cumbersome, uncomfortable and patients often will not continue to use these for prolonged periods of time. Weight loss may be effective but is rarely maintained by these patients.

In patients with central sleep apnea syndrome, phrenic nerve or diaphragmatic pacing has been used. Phrenic nerve or diaphragmatic pacing includes the use of electrical stimulation to regulate and control the patient's diaphragm which is innervated bilaterally by the phrenic nerves to assist or support ventilation. This pacing is disclosed in Direct Diaphragm Stimulation by J. Mugica et al. PACE vol. 10 January-February 1987, Part II, Preliminary Test of a Muscular Diaphragm Pacing System on Human Patients by J. Mugica et al. from Neurostimulation: An Overview 1985 pp. 263-279 and Electrical Activation of Respiration by Nochomovitez IEEE Eng. in Medicine and Biology; June, 1993.

However, it was found that many of these patients also have some degree of obstructive sleep apnea which worsens when the inspiratory force is augmented by the pacer. The ventilation induced by the activation of the diaphragm also collapses the upper airway upon inspiration and draws the patient's tongue inferiorly down the throat choking the patient. These patients then require tracheostomies for adequate treatment.

A physiological laryngeal pacemaker as described in Physiological Laryngeal Pacemaker by F. Kaneko et al. from Trans Am Soc Artif Intern Organs 1985, senses volume displaced by the lungs and stimulates the appropriate nerve to open the patient's glottis to treat dyspnea. This apparatus is not effective for treatment of sleep apnea. The apparatus produces a signal proportional in the displaced air volume of the lungs and thereby the signal produced is too late to be used as an indicator for the treatment of sleep apnea. There is often no displaced air volume in sleep apnea due to obstruction.

Surgical interventions have included uvulopalatopharyngoplasty, tonsillectomy, tracheostomy and surgery to correct severe retrognathia. One measure that is effective in obstructive sleep apnea is tracheostomy. However, this surgical intervention carries considerable morbidity and is aesthetically unacceptable to many patients. Other surgical procedures include a standard Le Fort I osteotomy in combination with a sagittal split ramus osteotomy. This is a major surgical intervention that requires the advancement of the maxilla, mandible and chin. Of the various surgical options available to the patient, this final procedure carries the longest recuperative period, accompanied by high cost and is the most invasive.

There is a need for a method and apparatus to treat airway obstruction disorders. There is a further need for a method and apparatus which delivers sufficient energy to an interior of a body structure, including but not limited to the tongue, to treat airway obstruction disorders while reducing a swelling of an exterior surface of the body structure.

### SUMMARY OF THE INVENTION

Accordingly, an object of the invention is to provide a method and apparatus to ablate interior regions of the tongue.

Another object of the invention is to provide a method and apparatus for ablating interior regions of the tongue without damaging the main branches of the hypoglossal nerves that control swallowing and speech functions.

These and other objects of the invention are achieved in an apparatus that reduces the volume of selected sections of a tongue. The apparatus includes an introducer means. An electrode means is at least partially positioned in an interior of the introducer means. The electrode means is configured to deliver sufficient energy to create cell necrosis in an interior of the tongue without damaging a main branch of the hypoglossal nerve. An electrode advancement and retraction means is coupled to the electrode means to advance and retract at least a portion of the electrode means in and out of a selected tongue surface. A cabling means is coupled to the energy delivery device means.

The introducer means includes an introducer tissue interface surface that can control a tongue surface temperature in the range of 10 to 45 degrees C. An energy delivery device may be hollow and coupled to an infusion medium source. An insulation sleeve can be positioned in a surrounding relationship to at least a portion of an exterior surface of the energy delivery device. One or more energy delivery devices may be introduced through the introducer. Additionally, one or more sensors can be positioned at various locations of the cell necrosis apparatus including at a distal end of the energy delivery device, at an exterior surface of the energy delivery device, at a distal end of the insulation sleeve or at the introducer tissue interface surface. A variety of different energy delivery device sources can be coupled to the energy delivery device including but not limited to RF.

In various embodiments the energy delivery device is introduced through the tongue's ventral surface, dorsal surface or the dorsum surface. Cell necrosis is achieved without damaging a main branch of the hypoglossal nerve.

### BRIEF DESCRIPTION OF THE FIGURES

FIGS. 1A-1C are cross-sectional views of a cell necrosis apparatus used with the present invention.

FIG. 2 is cross-sectional view illustrating the introducer and connector of the cell necrosis apparatus shown in FIGS. 1A-1C.

FIG. 3 is a perspective view of the connector illustrated in FIGS. 1A-1C.

FIGS. 4A-4C are perspective views of a needle electrode associated with the cell necrosis apparatus illustrated in FIGS. 1A-1C.

FIG. 5 is a perspective view of a flexible needle electrode utilized with the methods of the present invention.

FIG. 6 illustrates the creation of cell necrosis zones with the cell necrosis apparatus shown in FIGS. 1A-1C.

FIG. 7 is a cross-sectional view of the tongue with the mouth closed.

FIG. 8 is a cross-sectional view of the tongue with the mouth open.

FIG. 9 is a perspective view of the tongue.

FIG. 10 is a perspective view of the dorsum of the tongue.

FIG. 11 is a cross-sectional view of the tongue.

FIG. 12 is a cross-sectional view of the tongue illustrating the location of the main branches of the hypoglossal nerve and the creation of an cell necrosis zone.

FIG. 13 is a cross-sectional view of the tongue illustrating a plurality of cell necrosis zones.

FIG. 14 is a perspective view of the ventral surface of the tongue.

FIG. 15 is a cross-sectional view of the tongue.

FIG. 16 is a block diagram of a feedback control system useful with the methods of the present invention.

FIG. 17 is a block diagram illustrating an analog amplifier, analog multiplexer and microprocessor used with the feedback control system of FIG. 16.

FIG. 18 is a block diagram of a temperature/impedance feedback system that can be used to control cooling medium flow rate through the introducer of FIGS. 1A-1C.

### DETAILED DESCRIPTION

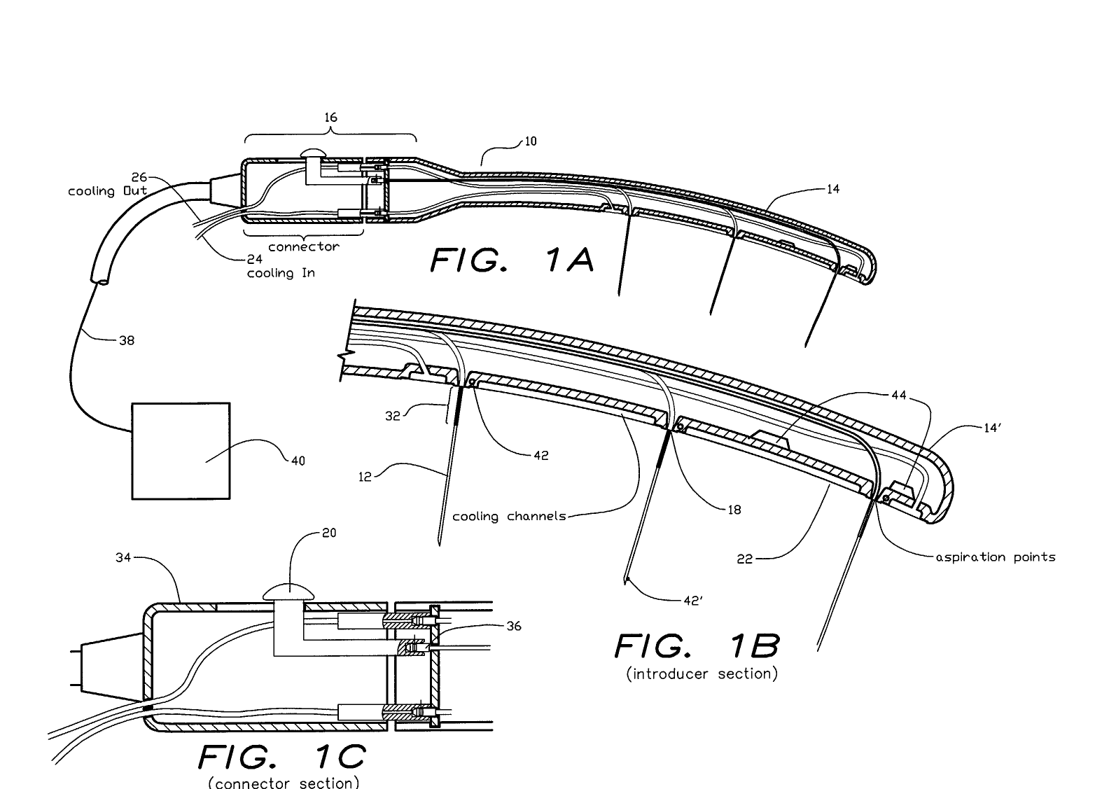

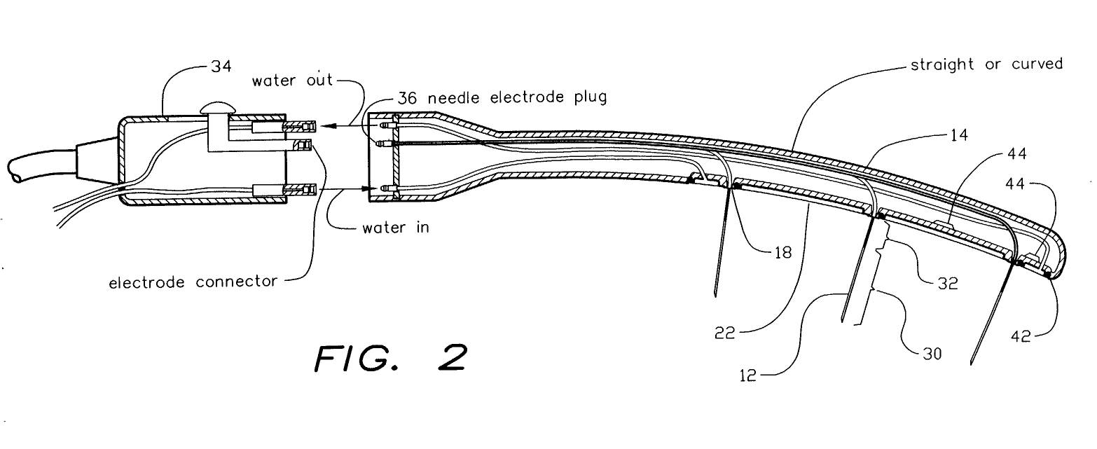

Referring to FIGS. 1A-1C and 2, a cell necrosis apparatus 10 for ablating the tongue, lingual tonsils, and/or adenoids is illustrated. For purposes of this disclosure, an ablation procedure shall be meant to include thermal damage, tissue shrinkage, tissue scarring, remodeling, debulking and causing ablation. Ablation apparatus 10 can be positioned so that one or more energy delivery devices 12 are introduced in an interior of the tongue through a surface of the tongue. Ablation apparatus 10 may include atraumatic intubation with or without visualization, provide for the delivery of oxygen or anesthetics, and can be capable of suctioning blood or other secretions. It will be appreciated that cell necrosis apparatus 10 is used to treat a variety of different obstructions in the body where passage of gas is restricted. One application is the treatment of sleep apnea using energy delivery devices 12 to ablate selected portions of the tongue, lingual tonsils and/or adenoids by the use of RF, microwave, ultrasound, coherent light, incoherent light, thermal transfer, resistive heating, chemical ablation, cryogenic fluid, electrolytic solutions and the like. In this regard, ablation apparatus 10 can be used to ablate targeted masses including but not limited to the tongue, tonsils, turbinates, soft palate tissues, uvula, hard tissue and, in selected instances, mucosal tissue. In one embodiment, ablation apparatus 10 is used to ablate an interior region of the tongue, causing it to become debulked, shrunk, remodeled, or to include tissue scarring in order to increase the cross-sectional area of the airway passage.

Prior to ablating the tongue, a presurgical evaluation may be performed including a physical examination, fiber optic pharyngoscopy, cephalometric analysis and polygraphic monitoring. The physical examination emphasizes the evaluation of the head and neck. It also includes a close examination of the nasal cavity to identify obstructing deformities of the septum and turbinate; oropharyngeal obstruction from a long, redundant soft palate or hypertrophic tonsils; and hypopharyngeal obstruction from a prominent base of the tongue.

Ablation apparatus 10 includes an introducer 14, a handle 16, one or more energy delivery devices 12 extending from different ports 18 formed along a longitudinal surface of introducer 14. An energy delivery device advancement and retraction device 20 is provided. Cabling is coupled to energy delivery devices 12. Introducer 14 and handle 16 may be one device.

Energy delivery devices 12 are at least partially positioned in an interior of introducer 14. Each energy delivery device 12 is advanced and retracted through a port 18 formed in an exterior surface of introducer 14. Energy delivery device advancement and retraction device 20 advances energy delivery devices 12 out of introducer 14, into an interior of a body structure and retracted back into introducer 14. Although the body structure can be any number of different structures, the body structure will hereafter be referred to as the tongue.

Energy delivery devices 12 pierce an exterior surface of the tongue and are directed to an interior region of the tongue. Sufficient energy is delivered by energy delivery devices 12 to the interior of the tongue to cause the tongue to become sufficiently ablated.

Energy delivery devices 12 can be hollow to receive a variety of different infusion mediums, including but not limited to saline solution. Energy delivery devices 12 may be limited in the distance that they can be advanced into the tongue. A means for limiting the travel of the energy delivery device 12 (which may be a needle electrode) may be provided within the handle 16. The limiting stop may be adjustable to provide variability in the amount of energy delivery devices 12 travel. This may be achieved with an insulation sleeve.

Energy delivery devices 12 can include a central lumen for receiving a variety of fluids that can be introduced into the interior of the tongue, as well as a plurality of fluid delivery ports. One suitable fluid is an electrolytic solution which can be used to enhance the delivery of energy from energy delivery device 12. Energy delivery can be direct from energy delivery device 12 to tissue, indirect from energy delivery device 12 to electrolytic solution to tissue, or a combination of the two. Another suitable fluid is a temperature control fluid which controls a tongue surface temperature in the range of 10-45 degrees C.

Introducer 14 may include an introducer tissue interface surface 22, a temperature control medium inlet conduit 24 and a temperature control medium outlet conduit 26 extending through an interior of introducer 14. Ports 18 are formed in the exterior wall of introducer 14, and are preferably formed on introducer tissue interface surface 22. Ports 18 are isolated from a temperature control medium that flows in inlet and outlet conduits 24 and 26.

Temperature control medium inlet and outlet conduits 24 and 26 are configured to provide a temperature controlled section of introducer tissue interface surface 22 of a radius of at least 1 to 2 cm² from port 18. More preferably, a temperature control section of introducer tissue interface surface 22 is at least equal to the cross-sectional diameter of the underlying zone of the ablation area.

The sizes of the temperature control sections are sufficient to minimize swelling of the tongue following the delivery of energy The reduction of swelling can be 50% or greater, 75% or greater, and 90% and greater. The amount of temperature control provided is sufficient to enable the patient to return home shortly after the ablation procedure is performed. This reduces the risk of choking on the tongue due to its swelling. It has been found that by providing a sufficient level of temperature control over a relatively large area, the amount of ablation in an interior region of the tongue is enhanced without increasing thermal damage at the surface of the tongue. This preserves the senses of taste and touch. By providing a large enough temperature controlled section of introducer tissue interface surface 22, an edematous response is minimized.

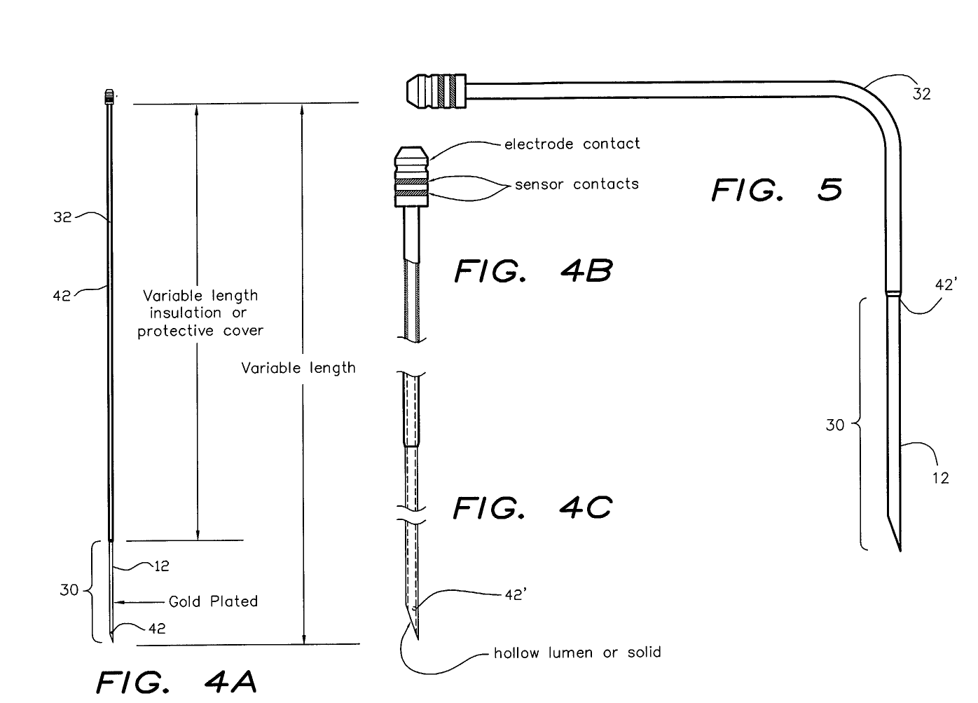

An energy delivery device surface 30 of energy delivery device 12 can be adjusted by inclusion of an adjustable or non-adjustable insulation sleeve 32 (FIGS. 4A-4C and 5). Insulation sleeve 32 can be advanced and retracted along the exterior surface of energy delivery device 12 in order to increase or decrease the length of the energy delivery device surface 30. Insulation sleeve 32 can be made of a variety of materials including but not limited to nylon, polyimides, other thermoplastics and the like.

The size of energy delivery device surface 30 can be varied by other methods including but not limited to creating a segmented energy delivery device 12. Additionally, a plurality of energy delivery devices 12 can be multiplexed and individually activated, and the like.

Handle 16 is preferably made of a thermal and electrical insulating material. Energy delivery devices 12 are made of a conductive material such as stainless steel. Additionally, energy delivery devices 12 can be made of a shaped memory metal, such as nickel titanium. In one embodiment, only a distal end of energy delivery device 12 is made of the shaped memory metal in order to effect a desired deflection. When introduced into the oral cavity, introducer 14 can be advanced until a patient's gag response is initiated. Introducer 14 is then retracted back to prevent patient's gagging. The distal end of energy delivery device 12 can be semi-curved. The distal end can also have a geometry to conform to an exterior of the tongue.

Introducer 14 may be malleable in order to conform to the surface of the tongue when a selected ablation target site is selected. A soft metal member may be enclosed or encapsulated within a flexible outer housing to form malleable introducer 14.

For many applications it is desirable for a distal end 14′ of introducer 14 to be conformable or deflectable. This can be achieved mechanically or with the use of memory metals. A steering wire, or other mechanical structure, can be attached to either the exterior or interior of distal end 14′ (FIGS. 1A-1C and 2). In one embodiment, a deflection knob located on handle 16 is activated by the physician causing a steering wire (not shown) to tighten. This imparts a retraction of distal end 14′, resulting in its deflection. It will be appreciated that other mechanical devices can be used in place of the steering wire. The deflection may be useful for tissue sites with difficult access.

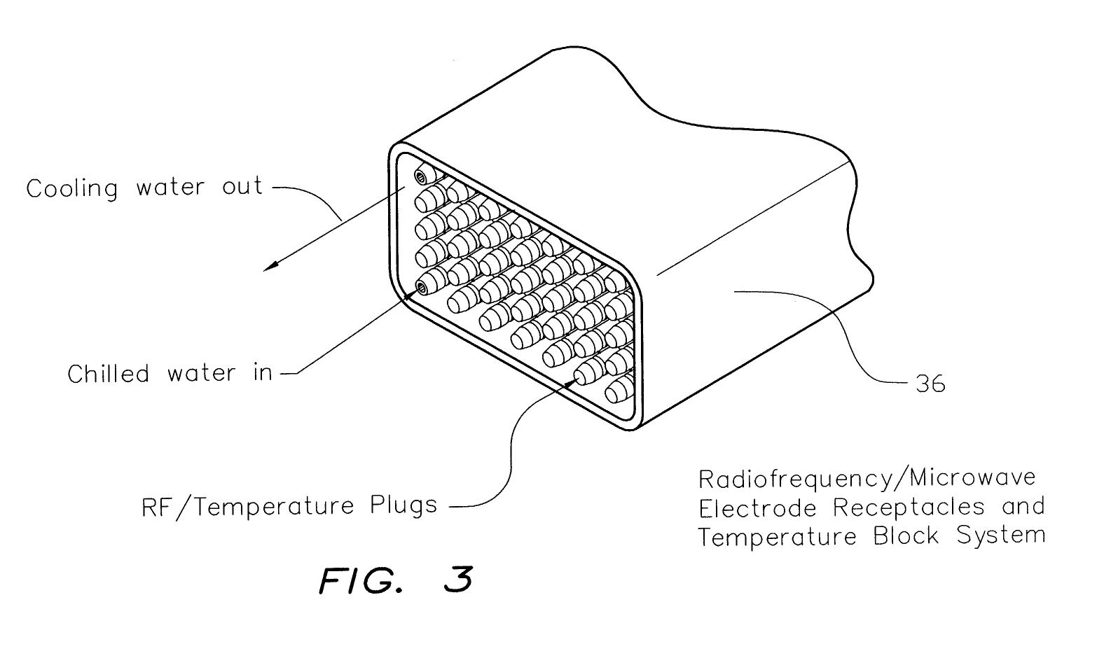

Handle 16 can include a connector 34 coupled to retraction and advancement device 20. Connector 34 provides a coupling of energy delivery devices 12 to power, feedback control, temperature and/or imaging systems. An RF/temperature control block 36 can be included when the energy delivery device is an RF electrode (FIG. 3).

In one embodiment, the physician moves retraction and advancement device 20 in a direction toward a distal end of connector 34. Energy delivery devices 12 can be spring loaded. When retraction and advancement device 20 is moved back, springs cause selected energy delivery devices 12 to advance out of introducer 14.

One or more cables 38 couple energy delivery device 12 to an energy source 40. A variety of energy sources 40 can be used with the present invention to transfer energy to the interior of a body structure, including but not limited to RF, microwave, ultrasound, coherent light, incoherent light, thermal transfer, resistive heating, chemical ablation, cryogenic fluid, electrolytic solutions and the like. Preferably, energy source 40 is an RF generator. When an RF energy source is used, the physician can activate RF energy source 40 by the use of a foot switch (not shown) coupled to RF energy source 40. Energy delivery device 12 may be a needle electrode.

One or more sensors 42 may be used to measure temperatures. For purposes of this specification, sensors which are not introduced into an interior of a body structure are denoted as 42. Sensors which are introduced into the body structure are denoted as 42′.

One or more sensors 42 and 42′ may be positioned on an interior or exterior surface of energy delivery device 12, insulation sleeve 32, or be independently inserted into the interior of the body structure. Sensors 42 and 42′ permit accurate measurement of temperature at a tissue site and if a predetermined maximum temperature is exceeded, the energy power supply/controller will reduce or shut down the power being delivered. By monitoring the temperature and modulating the energy delivered, sensors 42 and 42′ prevent non-targeted tissue from being destroyed or ablated.

Sensors 42 and 42′ are of conventional design, including but not limited to thermistors, thermocouples, resistive wires, and the like. Suitable sensors 42 include a T type thermocouple with copper constantan, J type, E type, K type, fiber optics, resistive wires, thermocouple IR detectors, and the like. It will be appreciated that sensors 42 and 42′ need not be thermal sensors.

By monitoring, sensors 42′ can measure the temperature at various points within the interior of the body structure. The data collected may be used to determine the temperature attained and by comparing the rate of rise against time, power level and impedance, the size and extent of lesion may be computed.

If at any time sensors 42 and 42′ determine that a desired temperature is exceeded, then an appropriate feedback signal is received at energy source 40 and the amount of energy delivered is regulated.

Ablation apparatus 10 can include visualization capability including but not limited to a viewing scope, ultrasound, an expanded eyepiece, fiber optics, video imaging, and the like.

Additionally, an ultrasound transducer 44 can determine the size and position of the created lesion. In one embodiment, two ultrasound transducers are positioned on opposite sides of introducer 14 to create an image depicting the lesion in the tongue. Each ultrasound transducer 44 is coupled to an ultrasound source (not shown).

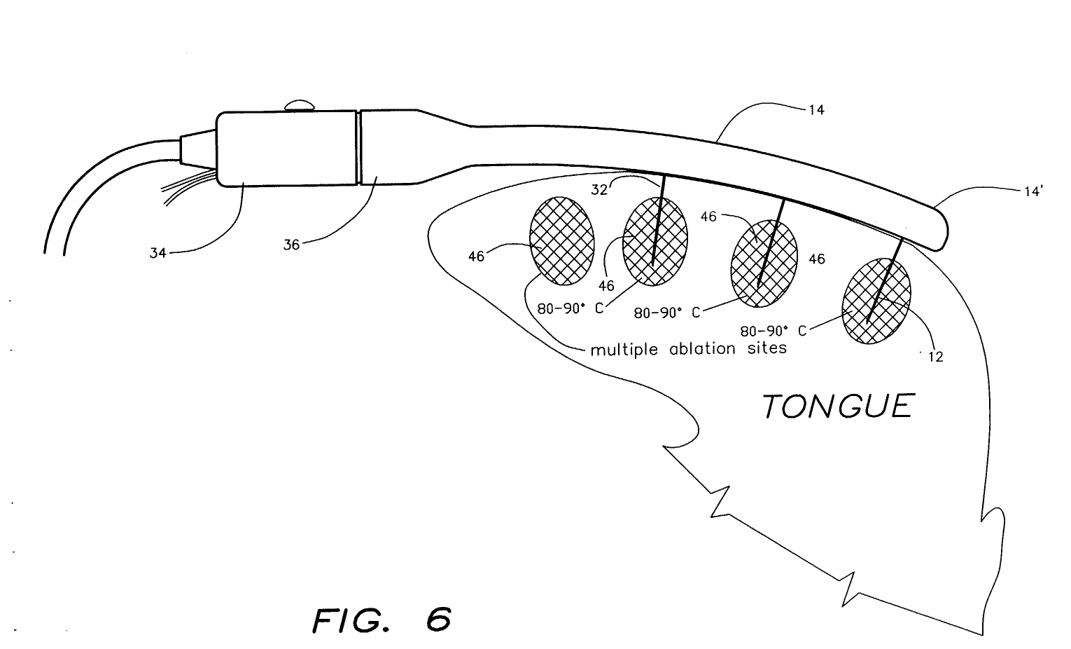

With reference now to FIG. 6 introducer 14 is shown as being introduced into the oral cavity and multiple RF electrodes 12 are advanced into the interior of the tongue creating different ablation zones 46. Ablation apparatus 10 can be operated in either bipolar or monopolar modes (with a ground pad). Electrodes 12 are operated in either mode to create ablation zones 46 in the tongue without damaging the main branches of the hypoglossal nerve. A larger airway passage is created. For purposes of this specification, the main branches of the hypoglossal nerve are those branches which if damaged create an impairment, either partial or full, of speech or swallowing capabilities. Creation of the ablation zone in the tongue results in a shrinkage of tissue, reshapes the posterior surface of the tongue, and debulks the tongue. The result is an increase in the cross-sectional diameter of the air passageway.

In one embodiment, a single electrode 12 is positioned in the tongue to create a first cell necrosis zone 46. Electrode 12 can then be retracted from the interior of the tongue, introducer 14 moved, and electrode 12 is then advanced from introducer 14 into another interior section of the tongue. A second cell necrosis zone 46 is created. This procedure can be completed any number of times to form different ablation regions in the interior of the tongue. Electrodes 12 are then repositioned in the interior of the tongue any number of times to create a plurality of connecting or non-connecting cell necrosis zones 46 in either bipolar or monopolar modes.

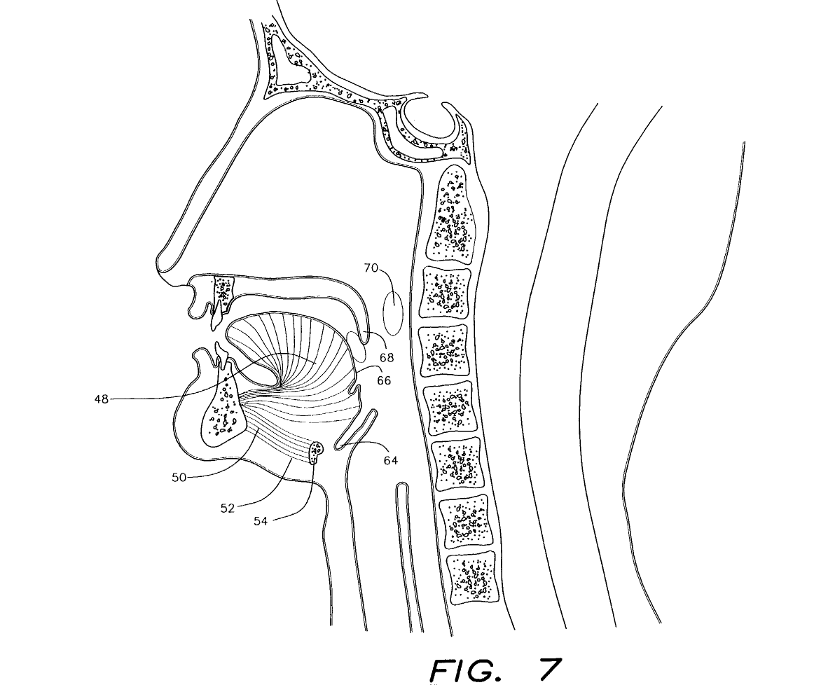

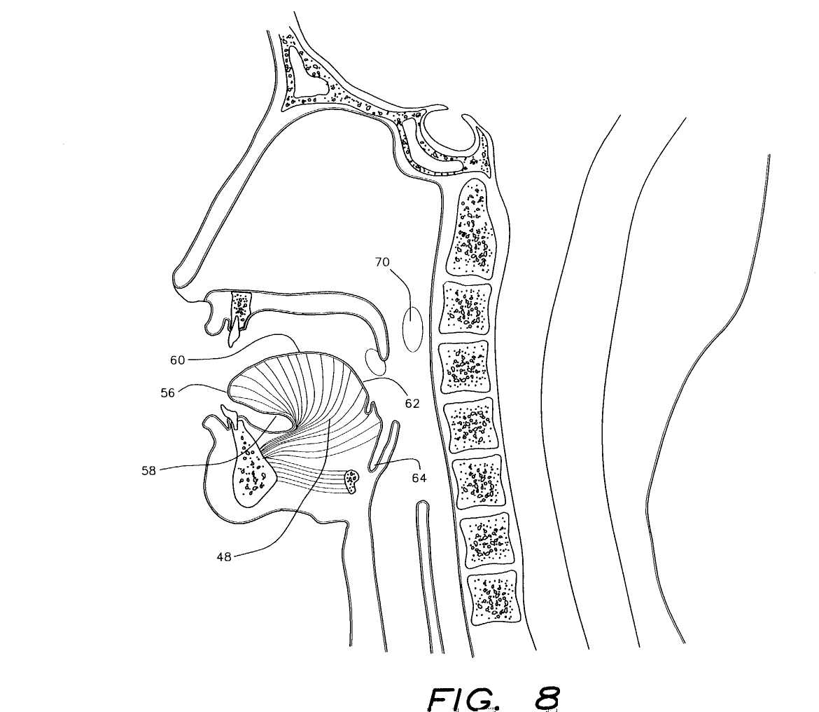

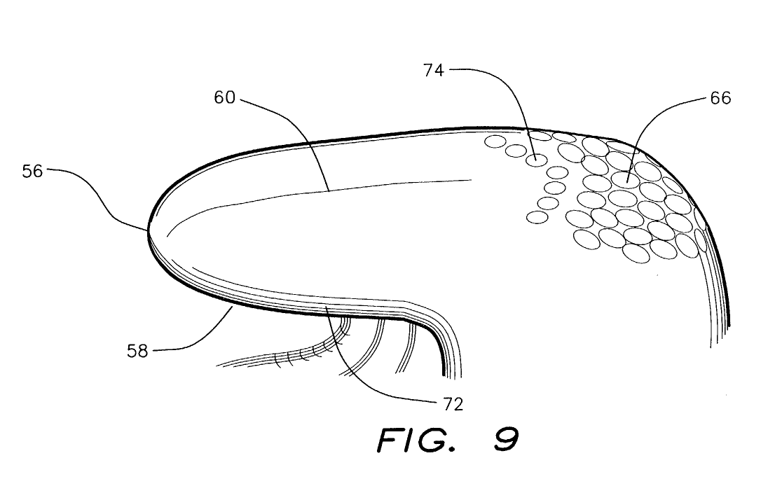

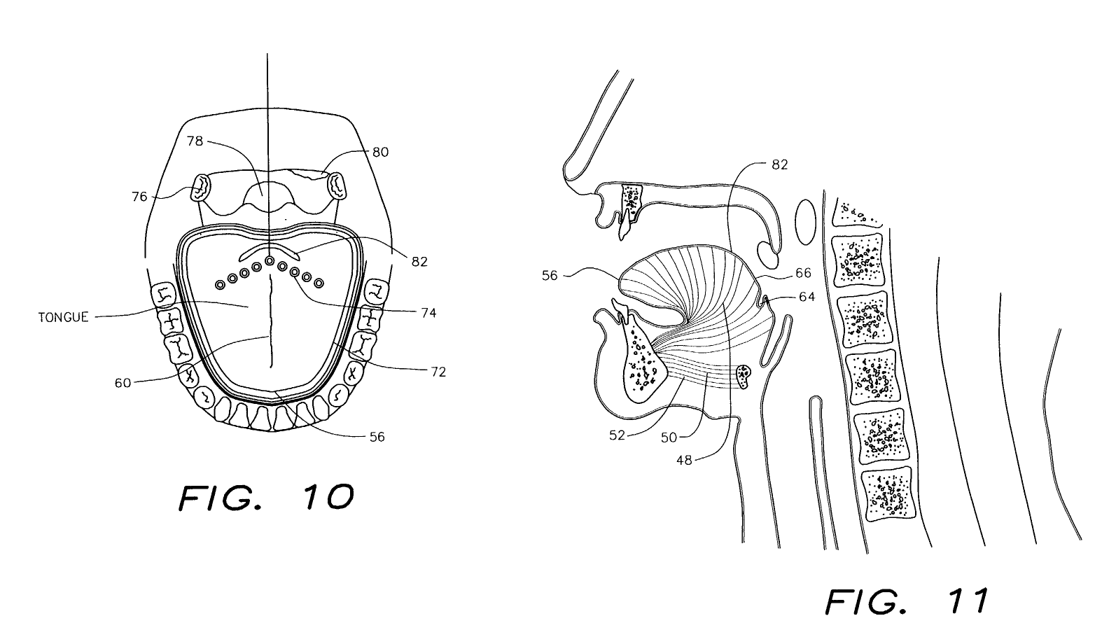

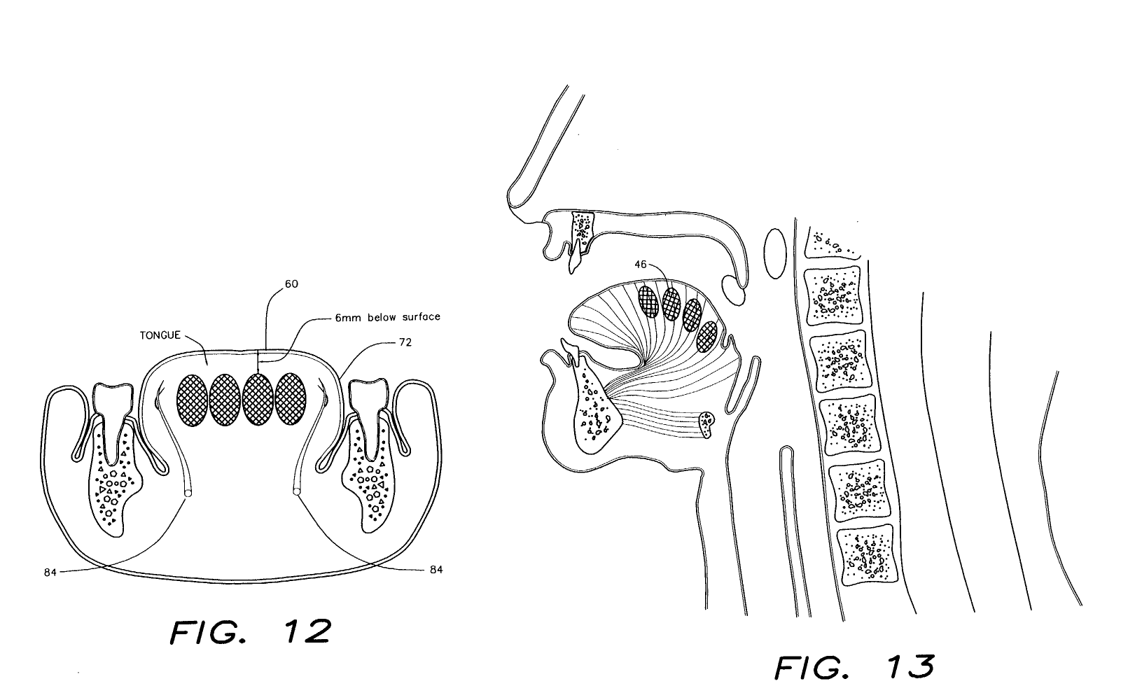

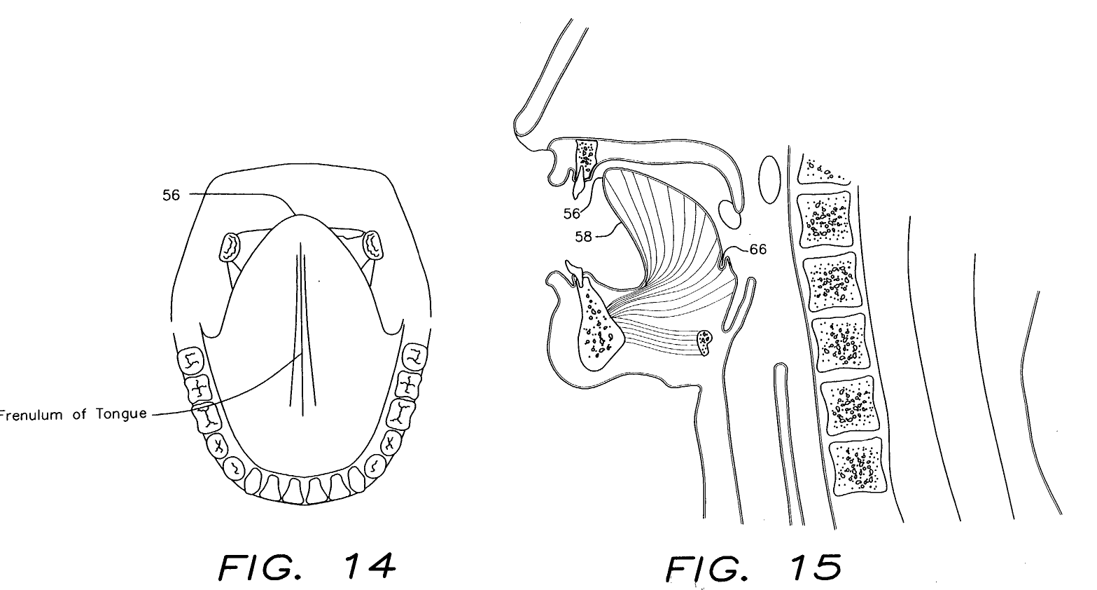

Referring now to FIGS. 7 through 15, various anatomical views of the tongue and other structures are illustrated. The different anatomical structures are as follows: the genioglossus muscle, or body of the tongue is denoted as 48; the geniohyoid muscle is 50; the mylohyoid muscle is 52; the hyoid bone is 54; the tip of the tongue is 56; the ventral surface of the tongue is denoted as 58; the dorsum of the tongue is denoted as 60; the inferior dorsal of the tongue is denoted as 62; the reflex of the vallecula is 64; the lingual follicles are denoted as 66; the uvula is 68; the adenoid area is 70; the lateral border of the tongue is 72; the circumvallate papilla is 74, the palatine tonsil is 76; the pharynx is 78; the redundant pharyngeal tissue is 80; the foramen cecum is 82; the main branches of the hypoglossal nerve are 84, and the lingual frenum of the tongue is 86.

Dorsum 60 is divided into an anterior 2/3 and inferior dorsal 62. The delineation is determined by circumvallate papilla 74 and foramen cecum 82. Inferior dorsal 62 is the dorsal surface inferior to circumvallate papilla 74 and superior reflex of the vallecula 64. Reflex of the vallecula 64 is the deepest portion of the surface of the tongue contiguous with the epiglottis. Lingual follicles 66 comprise the lingual tonsil.

Energy delivery devices 12 can be inserted into an interior of the tongue through dorsum surface 60, inferior dorsal surface 62, ventral surface 58, tip 56 or geniohyoid muscle 50. Additionally, energy delivery devices 12 may be introduced into an interior of lingual follicles 66 and into adenoid area 70. In all instances, the positioning of energy delivery device 12, as well as the creation of ablation zones 46 is such that the main branches of the hypoglossal nerve 84 are not ablated or damaged. The ability to swallow and speak is not impaired. This creates a larger air way passage and provides a treatment for sleep apnea.

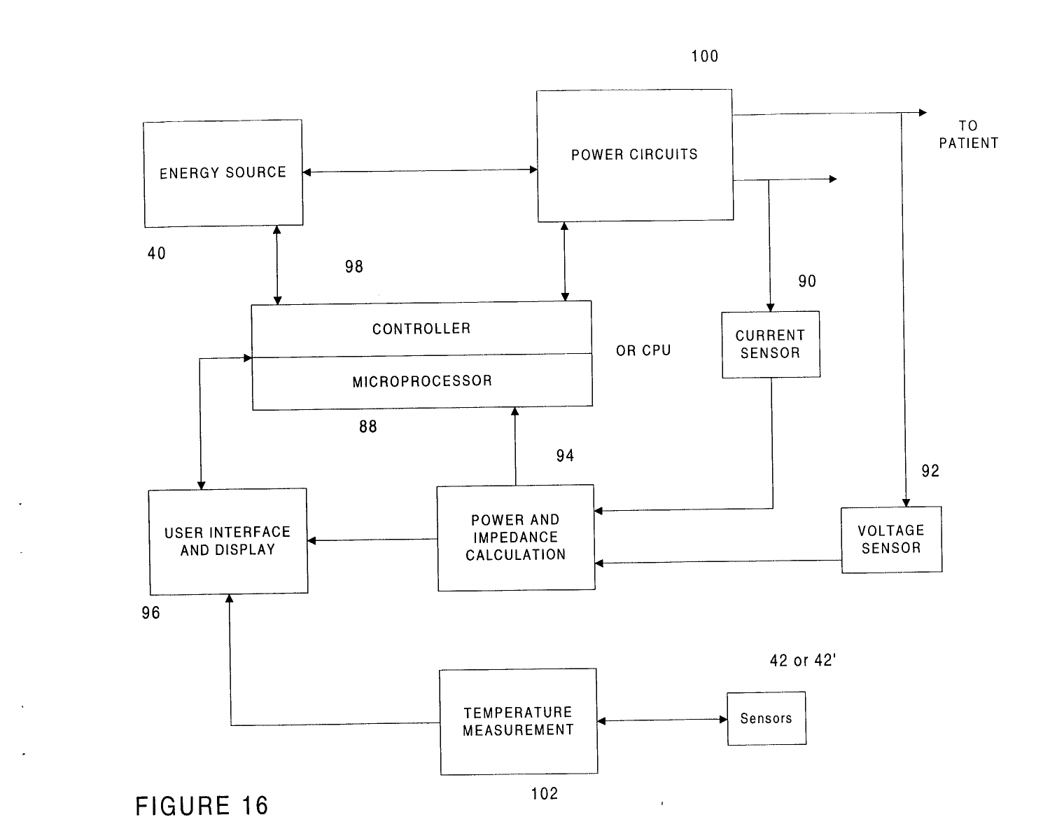

Referring now to FIG. 16, an open or closed loop feedback system couples sensors 42 or 42′ to energy source 40. In this embodiment, energy delivery device 12 is one or more RF electrodes. It will be appreciated that other energy delivery devices 12 can also be used with the feedback system.

The temperature of the tissue, or of RF electrode 12 is monitored, and the output power of energy source 40 adjusted accordingly. The physician can, if desired, override the closed or open loop system. A microprocessor can be included and incorporated in the closed or open loop system to switch power on and off, as well as modulate the power. The closed loop system utilizes a microprocessor 88 to serve as a controller, monitor the temperature, adjust the RF power, analyze the result, refeed the result, and then modulate the power.

With the use of sensors 42′ and the feedback control system a tissue adjacent to energy delivery device can be maintained at a desired temperature for a selected period of time without impeding out. Each RF electrode 12 is connected to resources which generate an independent output. The output maintains a selected energy at RF electrodes 12 for a selected length of time.

Current delivered through RF electrodes 12 is measured by current sensor 90. Voltage is measured by voltage sensor 92. Impedance and power are then calculated at power and impedance calculation device 94. These values can then be displayed at user interface and display 96. Signals representative of power and impedance values are received by a controller 98.

A control signal is generated by controller 98 that is proportional to the difference between an actual measured value, and a desired value. The control signal is used by power circuits 100 to adjust the power output in an appropriate amount in order to maintain the desired power delivered at respective RF electrodes 12.

In a similar manner, temperatures detected at sensors 42′ provide feedback for maintaining a selected power. Temperature at sensors 42 are used as safety devices to interrupt the delivery of energy when maximum pre-set temperatures are exceeded. The actual temperatures are measured at temperature measurement device 102, and the temperatures are displayed at user interface and display 96. A control signal is generated by controller 98 that is proportional to the difference between an actual measured temperature and a desired temperature. The control signal is used by power circuits 100 to adjust the power output in an appropriate amount in order to maintain the desired temperature delivered at the respective sensor 42 or 42′. A multiplexer can be included to measure current, voltage and temperature, at the numerous sensors 42 and 42′, and energy can be delivered to RF electrodes 12 in monopolar or bipolar fashion.

Controller 98 can be a digital or analog controller, or a computer with software. When controller 98 is a computer it can include a CPU coupled through a system bus. On this system can be a keyboard, a disk drive, or other non-volatile memory systems, a display, and other peripherals, as are known in the art. Also coupled to the bus is a program memory and a data memory.

User interface and display 96 includes operator controls and a display. Controller 98 can be coupled to imaging systems, including but not limited to ultrasound, CT scanners, X-ray, MRI, mammographic X-ray and the like. Further, direct visualization and tactile imaging can be utilized.

The output of current sensor 90 and voltage sensor 92 is used by controller 98 to maintain a selected power level at RF electrodes 12. The amount of RF energy delivered controls the amount of power. A profile of power delivered can be incorporated in controller 98 and a preset amount of energy to be delivered may also be profiled.

Circuitry, software and feedback to controller 98 result in process control, and the maintenance of the selected power setting that is independent of changes in voltage or current, and are used to change, (i) the selected power setting, (ii) the duty cycle (on-off time), (iii) bipolar or monopolar energy delivery and (iv) fluid delivery, including flow rate and pressure. These process variables are controlled and varied, while maintaining the desired delivery of power independent of changes in voltage or current, based on temperatures monitored at sensors 42 or 42′

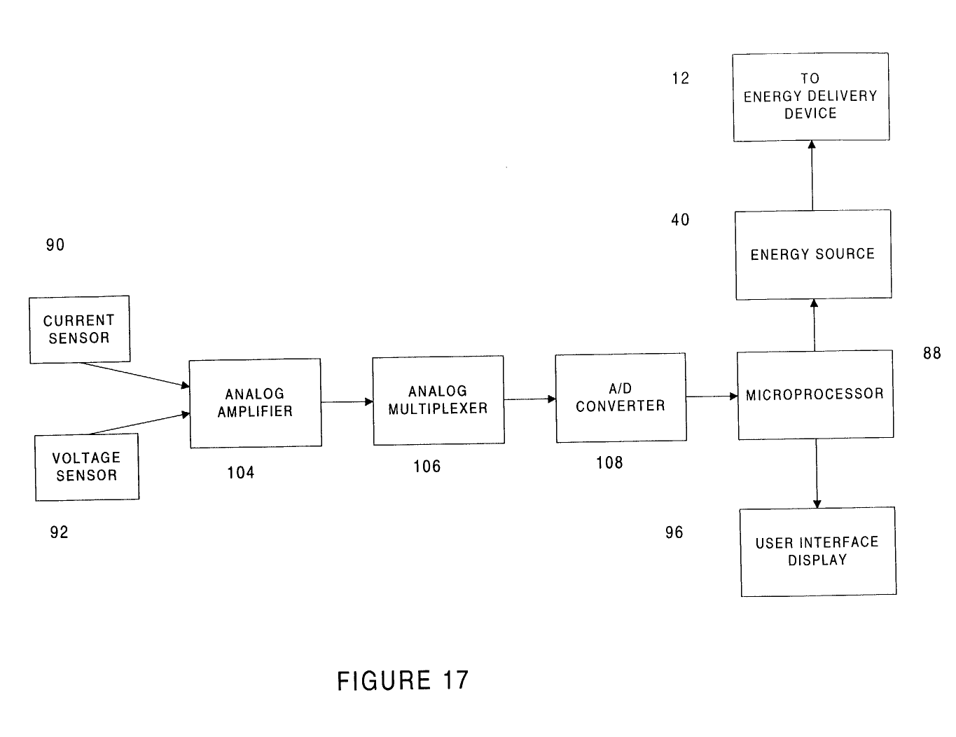

Referring now to FIG. 17, current sensor 90 and voltage sensor 92 are connected to the input of an analog amplifier 104. Analog amplifier 104 can be a conventional differential amplifier circuit for use with sensors 42 and 42′. The output of analog amplifier 104 is sequentially connected by an analog multiplexer 106 to the input of A/D converter 108. The output of analog amplifier 104 is a voltage which represents the respective sensed temperatures. Digitized amplifier output voltages are supplied by A/D converter 108 to microprocessor 88. Microprocessor 88 may be a type 68HCII available from Motorola. However, it will be appreciated that any suitable microprocessor or general purpose digital or analog computer can be used to calculate impedance or temperature.

Microprocessor 88 sequentially receives and stores digital representations of impedance and temperature. Each digital value received by microprocessor 88 corresponds to different temperatures and impedances.

Calculated power and impedance values can be indicated on user interface and display 96. Alternatively, or in addition to the numerical indication of power or impedance, calculated impedance and power values can be compared by microprocessor 88 with power and impedance limits. When the values exceed predetermined power or impedance values, a warning can be given on user interface and display 96, and additionally, the delivery of RF energy can be reduced, modified or interrupted. A control signal from microprocessor 88 can modify the power level supplied by energy source 40.

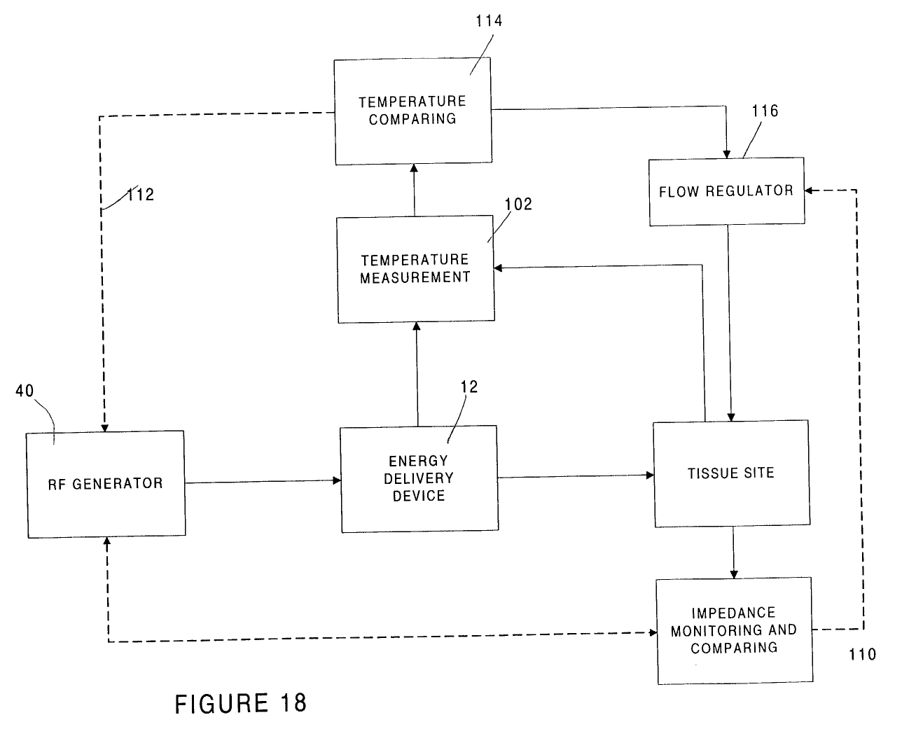

FIG. 18 illustrates a block diagram of a temperature/impedance feedback system that can be used to control temperature control fluid flow rate through introducer 14. Energy is delivered to RF electrode 12 by energy source 40, and applied to tissue. A monitor 110 ascertains tissue impedance, based on the energy delivered to tissue, and compares the measured impedance value to a set value. If the measured impedance exceeds the set value a disabling signal 112 is transmitted to energy source 40, ceasing further delivery of energy to RF electrodes 12. If measured impedance is within acceptable limits, energy continues to be applied to the tissue. During the application of energy to sensor 42 and 42′ measures the temperature of tissue and/or electrode 12. A comparator 114 receives a signal representative of the measured temperature and compares this value to a pre-set signal representative of the desired temperature. Comparator 114 sends a signal to a flow regulator 116 representing a need for a higher temperature control fluid flow rate, if the tissue temperature is too high, or to maintain the flow rate if the temperature has not exceeded the desired temperature.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. An apparatus for reducing the volume of selected sections of a tongue, comprising:
an introducer means;
an energy delivery device means at least partially positioned in an interior of the introducer means, the energy delivery device means being configured to deliver sufficient energy to ablate an interior of the tongue without damaging a main branch of the hypoglossal nerve of the tongue;
an energy delivery device advancement and retraction means coupled to the energy delivery device means to advance and retract at least a portion of the energy delivery device means in and out of a selected tongue surface; and
a cabling means coupled to the energy delivery device means.

2. The apparatus of claim 1, further comprising:
an energy source means coupled to the energy delivery device means and the cabling means.

3. The apparatus of claim 1, further comprising:
a cooling means at least partially positioned in the interior of the introducer means and configured to cool a surface of the tongue.

4. The apparatus of claim 3, further comprising:
means for controlling a cooling medium flow rate through the cooling means.

5. The apparatus of claim 1, further comprising:
an insulator means positioned at least partially around an exterior of the energy delivery device means.

6. The apparatus of claim 5, further comprising:
a sensor means positioned at a distal end of the insulator means.

7. The apparatus of claim 1, further comprising:
a sensor means positioned at a distal end of the energy delivery device means.

8. The apparatus of claim 1, further comprising:
a sensor means positioned on an exterior of the introducer means.

9. The apparatus of claim 1, further comprising:
a first sensor means positioned at a distal end of the energy delivery device means and a second sensor means positioned at a distal end of an insulator means positioned at least partially around an exterior of the energy delivery device means.

10. The apparatus of claim 1, wherein the energy delivery device means is a RF electrode coupled to a RF generator.

11. The apparatus of claim 1, wherein the energy delivery device means is a microwave antenna coupled to a microwave source.

12. The apparatus of claim 1, further comprising:
a feedback control means coupled to the energy delivery device means and an energy source means.

13. The apparatus of claim 12, further comprising:
an ultrasound means coupled to the feedback control means.

14. The apparatus of claim 1, wherein the energy delivery device means includes two or more RF electrodes means coupled to an RF energy source means.

15. The apparatus of claim 1, further comprising:
an infusion medium source means coupled to the energy delivery device means.

16. The apparatus of claim 1, further comprising:
a temperature control means at least partially positioned in the interior of the introducer means and configured to monitor a surface of the tongue at a temperature of 34 degrees C. or higher.

17. The apparatus of claim 16, further comprising:
means for controlling a temperature control fluid flow rate through the temperature control means.

18. A method for reducing a volume of a tongue, comprising:
providing an ablation apparatus including one or more RF electrodes coupled to an RF energy source;
positioning at least one electrode into an interior of the tongue;
delivering RF energy from the electrode into the interior of the tongue; and
ablating a section in the interior of the tongue without damaging a main branch of the hypoglossal nerve.

19. A method for treating airway obstructions, comprising:
providing an ablation apparatus including one or more RF electrodes coupled to an RF energy source;
positioning at least one electrode into an interior of a tongue;
delivering RF energy from the electrode into the interior of the tongue; and
creating cell necrosis in the interior of the tongue without damaging a hypoglossal nerve.

20. A method for treating airway obstructions, comprising:
providing an ablation apparatus including one or more RF electrodes coupled to an RF energy;
positioning at least one electrode into an interior of a lingual tonsil;
delivering sufficient RF energy from the electrode into an interior of the lingual tonsil;
creating cell necrosis in the interior of the lingual tonsil.
